(19) United States
(12) Patent Application Publication
(10) Pub. No.: US 2023/0329617 A1
(43) Pub. Date: Oct. 19, 2023

(54) NEURAL NETWORK INTRACARDIAC EGM ANNOTATION

(71) Applicant: BIOSENSE WEBSTER (ISRAEL) LTD., Yokneam (IL)

(72) Inventors: Vadim Gliner, Haifa (IL); Assaf Govari, Haifa (IL)

(21) Appl. No.: 17/721,393

(22) Filed: Apr. 15, 2022

(51) Int. Cl. A61B 5/287; A61B 5/00; G06N 3/04

(52) U.S. Cl. CPC A61B 5/287; A61B 5/0006; G06N 3/04

(57) ABSTRACT

A method for medical diagnosis, consisting of receiving electrophysiological data including first intracardiac electrogram (IEGM) signals acquired by a first pair of electrodes having a first intracardiac electrode. The data includes second IEGM signals acquired by a second pair of electrodes having a second intracardiac electrode in proximity to the first intracardiac electrode. The first and second IEGM signals are input to a first convolutional layer of a neural network and a second convolutional layer parallel to the first convolutional layer in the neural network to generate respective first and second interim results. The first and second interim results are input into one or more common layers of the neural network. The method includes receiving from an output layer of the neural network, responsively to inputting the first and second interim results, an indication of a local activation time at a location of the first and second intracardiac electrodes.

### FIELD OF THE DISCLOSURE

This disclosure relates generally to electrocardiograms (EGMs), and specifically to annotation of intracardiac EGM signals.

### BACKGROUND

An annotation of an EGM signal is a label that points to a specific location within the signal. Methods for annotating EGM signals are known in the prior art, some of which are referenced below.

US Patent Application No. 2021/0386355, to Ravuna et al., describes a method for detecting a mapping annotation for an electrophysiological (EP) mapping system. The method includes using a processor having a machine learning algorithm that is configured to receive a first heartbeat at an identified cardiac spatial location.

US Patent Application No. 2022/0005198, to Goldberg et al., describes a method that is provided by an ablation contiguity engine executed by a processor. The ablation contiguity engine is stated to utilize manual annotations of training data.

Chinese Patent Application CN111096735A, to Ou Feng et al., describes an electrocardiogram analysis system that is capable of being updated iteratively. The system is stated to have a back-end processing center that may include an annotation data expansion module.

European Patent Application EP3865060A1, to Amaury et al., describes a computer-implemented method for generating an annotated photoplethysmography (ppg) signal. The method is stated to include recording of an ECG signal and annotating segments in the ECG signal either algorithm-based or expert-based.

PCT application WO2020/214094A1, to Bhograjet al., describes a system for facilitating data processing of physiological information. The system is stated to comprise a device that is operable to collect a dataset of physiological information from a person, and a server operable to receive the dataset from the device and classify the dataset into at least one preliminary group of a medical condition.

### BRIEF DESCRIPTION OF THE DRAWINGS

The foregoing and other features and advantages of the invention will be apparent from the following, more particular description of preferred embodiments of the invention, as illustrated in the accompanying drawings.

FIG. 1 is a schematic pictorial illustration of an electrocardiogram (EGM) signal acquisition system used in a cardiac diagnostic procedure;

FIG. 2 is a schematic block diagram illustrating the overall structure and operation of an artificial neural network;

FIG. 3 is a schematic diagram of an example of a set of layers in the artificial neural network;

FIG. 4 is a schematic diagram of an alternative example of the set of layers;

FIG. 5 is a schematic diagram of an example of another set of layers;

FIG. 6 is a schematic diagram of an alternative example of the set of layers of FIG. 5;

FIG. 7 is a flowchart of an algorithm for generating a corpus of samples; and

FIG. 8 is a flowchart of an algorithm for training the artificial neural network of FIG. 2.

### DESCRIPTION OF EXAMPLES

### Overview

To map the flow of the electric wave as it passes through a beating heart of a patient, a physician may insert a catheter with an electrode into the heart during a diagnostic investigation of the heart. As the wave traverses the heart, it causes expansion and contraction of the region traversed, so that the heart beats. The inserted electrode detects the passage of the wave as an intracardiac electrocardiogram voltage vs. time signal, and the point in time at which the wave traverses a given region of the heart, termed the local activation time (LAT), is a parameter that the physician may use in diagnosing the heart condition.

Typically the catheter has two electrodes, and the voltage referred to above may be the voltage, herein termed a bipolar voltage or signal, between the two electrodes. In addition the patient may have an electrode, such as patch electrode attached to the patient's skin, that may be used as a reference, or a WCT (Wilson central terminal) reference may be used. Using one of these references, the voltage referred to above may be the voltage, herein termed a unipolar voltage or signal, measured relative to the reference. It will be understood that there is a unipolar voltage for each of the two electrodes.

An accepted method for automatically finding the LAT of a region is to use a wavefront annotation algorithm, described in U.S. Pat. No. 11,058,342 to Botzer et al. The algorithm determines the LAT by finding a point in time at which a unipolar voltage signal has its steepest descent. This time, which may be adjusted using the bipolar signal and other parameters of the signals, is typically set to be the LAT. Other methods for automatically finding the LAT of a region are also known in the art.

However, automatic determination of the LAT is not foolproof; for example, the signal analyzed may have more than one descent, there may be differences (which the algorithm used may not be able to account for) in the shape of the signals for different subjects, and/or there may be differences in the shape of the signal for different regions for one subject. The errors caused by the incorrect LAT determination may be corrected manually, typically by the physician performing the diagnostic investigation, and overriding the LAT value assigned automatically based on his/her experience.

Present-day catheters used for diagnosis may have many electrodes, often more than one hundred, with each electrode generating at least a unipolar signal and also typically being used for a bipolar signal. For these catheters manual correction of the large number of signals becomes burdensome and costly, since it relies upon the expertise of the physician.

Examples of the present disclosure improve on algorithmic prior art methods of finding the LAT, by using a trained artificial neural network having a plurality of parallel sets of layers to analyze the signals acquired by a pair of electrodes. In one example, there are four parallel sets of layers, respectively dedicated to analyzing the two unipolar signals, the bipolar signal, and a combination of the unipolar and bipolar signals. The results of each of the sets of layers are combined together in the network to give an overall estimate of the LAT of the signal.

To train the network requires extremely large numbers, of the order of one million or more, of signals to form a corpus of training data. While, theoretically, a corpus for training may be acquired from healthy patients, in practice most available data is from patients who are unhealthy. The data is available from unhealthy patients because they have undergone a diagnostic investigation; healthy patients typically do not undergo a diagnostic investigation. The problem with using data from unhealthy patients is that the LAT that is found automatically may be caused by either an unhealthy region in the heart, e.g., patient pathology such as a fibrotic region, or because of an error in the operation of the algorithm determining the LAT.

Examples of the present disclosure provide a method for generating the corpus of large amounts of data needed. Intracardiac EGM (IEGM) signals are acquired from a healthy animal, typically a pig. An algorithm such as the wave annotation algorithm referred to above is used to automatically calculate the LAT of each signal, and the resulting data may be presented, colored according to the time of the LAT, on a map of the heart. This method of presenting LATs is well known in the cardiology art.

Because the data acquired is from a healthy animal, the LAT problem referred to above—that the signal observed may be from an unhealthy region of the heart—does not exist. Thus, because in a healthy animal, LAT values change in a smooth, gradual, manner across the heart, the LAT colors presented in the map should also change in a smooth, gradual, manner, and any anomalies in this gradual color change are easily visible. For example, in a green region of the LAT map there may be a blue point; such a point corresponds to a signal where the algorithm calculating the LAT has found an anomalous LAT that is different from LAT values of its surroundings. However, because the signals are from healthy animals, the different color must be because of an algorithm error.

An inspector of the map may correct the anomalous LAT of the blue point by simply selecting the point, wherein a processor may automatically reassign the LAT of the point to a value corresponding to the surrounding LATs. Alternatively, the inspector may manually reassign the LAT. In addition, on selection the processor may save the signal and the reassigned value of the LAT, and this signal and its LAT may be added to the corpus of training data and may be used to train the network of the disclosure. It will be understood that the inspector selecting the signals to be corrected does not require the expertise of a physician.

### System Description

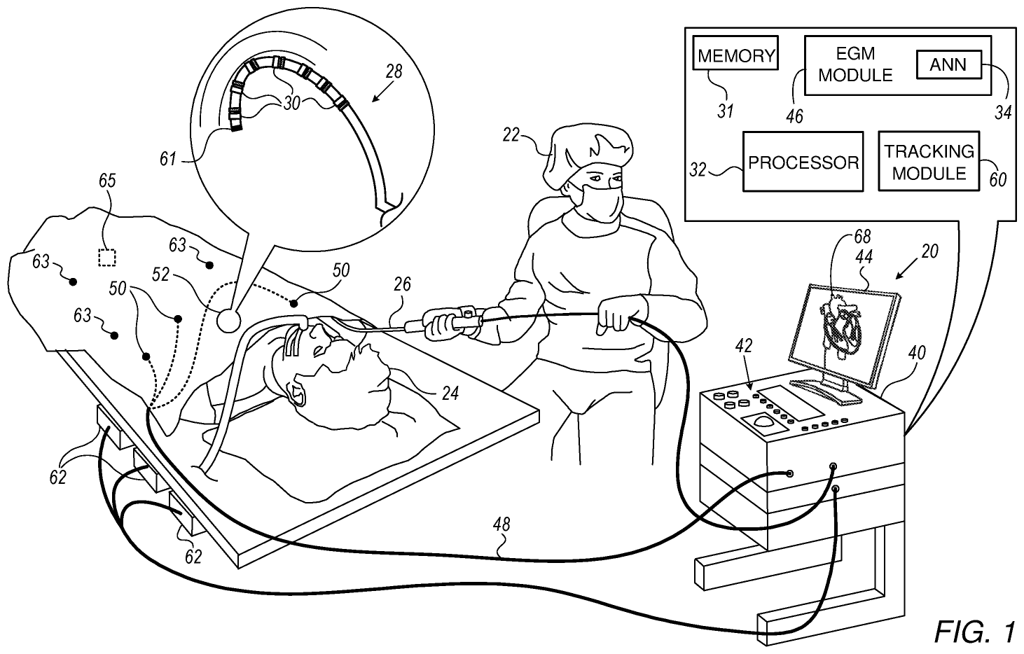

Reference is now made to FIG. 1, which is a schematic pictorial illustration of an electrocardiogram (EGM) signal acquisition system 20 used in a cardiac diagnostic procedure. In the pictured example, a physician 22 performs the diagnostic procedure using system 20. Physician 22 performs the procedure on a heart 52 of a subject 24, using a catheter probe 26 having a distal end 28 comprising multiple substantially similar electrodes 30 arranged along the length of the distal end. In the present disclosure electrodes 30 are differentiated, as necessary, by appending a suffix A, B, . . . to the identifying numeral.

System 20 comprises a processor 32, a memory 31, and an EGM module 46. EGM module 46 is coupled through a cable 48 to EGM electrodes 50, which are attached to the skin of subject 24 so as to acquire body surface EGM signals from the subject. EGM module 46 is also configured to acquire intracardiac EGM signals from electrodes 30 on distal end 28, and to analyze these signals using an artificial neural network (ANN) 34 incorporated in the EGM module. The operation of ANN 34 is described in more detail below.

Processor 32 and module 46 typically comprise a programmable processor, which is programmed in software and/or firmware to carry out the functions that are described herein. Alternatively or additionally, the processor may comprise hard-wired and/or programmable hardware logic circuits, which perform at least some of these functions. Although processor 32 and module 46 are shown in the figure, for the sake of simplicity, as separate, monolithic functional blocks, in practice some of these functions may be combined in a single processing and control unit.

Processor 32 and EGM module 46 typically reside within a console 40. Console 40 comprises input devices 42, such as a keyboard and a mouse, operated by physician 22. A display screen 44 is located in proximity to console 40, and may be used to present a map 68 of heart 52. Display screen 44 may optionally comprise a touch screen, thus providing another input device.

System 20 may comprise other modules, typically residing within console 40, for operation of the system.

For example, the system may comprise a tracking module 60 that is coupled to one or more electromagnetic position sensors 61 in distal end 28. In the presence of an external magnetic field generated by magnetic-field generators 62, the electromagnetic position sensors output signals that vary with the positions of the sensors. Based on these signals, tracking module 60 may ascertain the positions of electrodes 30 in heart 52.

A method of position tracking using external magnetic fields is implemented in the CARTO™ system, produced by Biosense Webster Inc. (Irvine, Calif.) and is described in detail in U.S. Pat. Nos. 5,391,199, 6,690,963, 6,484,118, 6,239,724, 6,618,612 and 6,332,089, in PCT Patent Publication WO 96/05768, and in U.S. Patent Application Publication Nos. 2002/0065455 A1, 2003/0120150 A1 and 2004/0068178 A1.

Alternatively or additionally, module 60 may use a tracking system based on currents transferred through, or impedances seen by, electrodes 30. In such a system module 60 estimates the position of a given electrode 30 in response to currents or impedances between the given electrode and a plurality of surface-electrodes 63 that are coupled to the skin of subject 24. An Advanced Current Location (ACL) system, made by Biosense-Webster (Irvine, California), which is described in U.S. Pat. No. 8,456,182, is such a tracking system.

An external electrode 65, or “return patch”, may be additionally attached externally to subject 24, typically on the skin of the subject's torso, for use as an indifferent or reference electrode. Alternately a reference electrode such as a Wilson central terminal (WCT) electrode may be formed by positioning electrodes 50 appropriately.

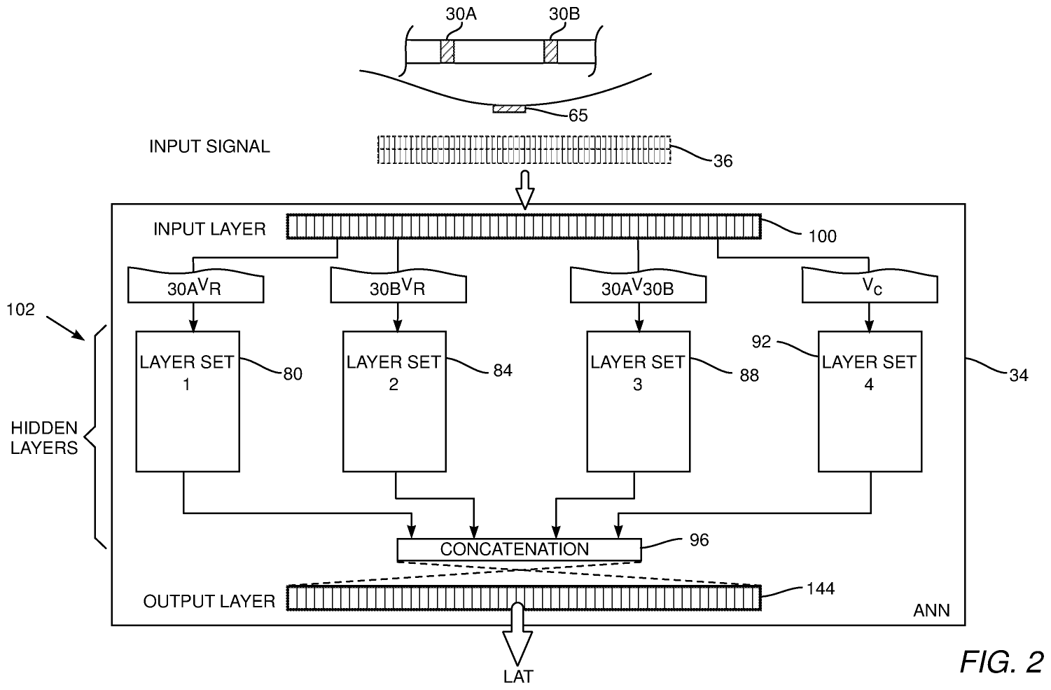

FIG. 2 is a schematic block diagram illustrating the overall structure and operation of ANN 34. In the illustrated figure, ANN 34, after training, is configured to receive as an input signal 36 unipolar and bipolar signals acquired by electrodes 30A and 30B measured with respect to each other and with respect to an indifferent reference electrode, by way of example assumed to comprise patch 65, and also referred to herein as reference R. Electrodes 30A and 30B are typically separated from each other by approximately 1 mm.

Signal 36 is acquired in a preset window of interest (WOI), and comprises a plurality of values. In the example described herein there are 401 digitized elements of the signal, each element being taken at a respective acquisition time point within the WOI. Typically signal time points are separated by, and have a “width” of, approximately 1 ms. It will be understood that this number of elements, and their separation, are by way of example, and other examples may have larger or smaller numbers of digitized elements of the signal and corresponding separations.

ANN 34 is formed of layers of artificial neurons, and hereinbelow each of the layers is assumed to comprise rectified linear unit (ReLU) neurons. However, the layers of ANN 80A may be comprised of other neurons, such as derivations of ReLU neurons, tanh neurons and/or sigmoid neurons, and those having ordinary skill in the art will be able to adapt the disclosure, mutatis mutandis, for layers with other such neurons.

In a first input layer 100 there are 401 neurons, corresponding to the number of elements, i.e., the number of time points, in input signal 36. Input layer 100 is followed by hidden layers 102 which are formed as a plurality of parallel sets of layers 80, 84, 88, and 92, each set of layers comprising at least one convolution layer. Prior to the procedure of FIG. 1, network 34, including all the sets of layers, has been trained, and the method of training, as well as the structure of each of the sets of layers, is described below.

Specifically, set 80 receives from input layer 100 a first unipolar signal 30A^{V}R measured between electrode 30A and reference R, set 84 receives from the input layer a second unipolar signal 30B^{V}R measured between electrode 30B and reference R, set 88 receives a bipolar signal 30AV30B measured between electrode 30A and electrode 30B, and set 92 receives a combination V_{C} of the signals 30A^{V}R, 30B^{V}R, 30AV30B The outputs of each of the sets of layers are combined in a concatenation layer 96, comprised within hidden layers 102. Concatenation layer 96 feeds to a fully connected output layer 144 which provides a final estimate of the LAT of the signal acquired from electrodes 30A, 30B.

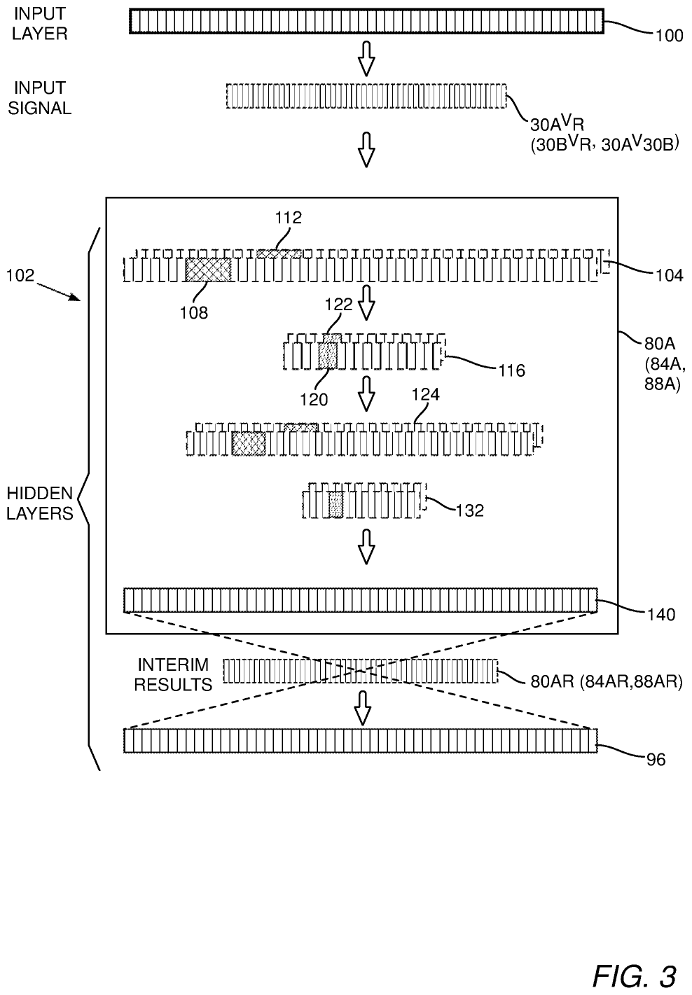

FIG. 3 is a schematic diagram of an example of set 80, herein termed set 80A, which is configured to receive as an input signal, from input layer 100, the 30A^{V}R unipolar signal acquired by electrode 30A. As stated above, in input layer 100 there are 401 neurons, corresponding to the number of elements, i.e., the number of time points, in the input signal 30A^{V}R. The 30A^{V}R acquired signal may be considered to be a 1-dimensional (1D) matrix in the form of a lxn matrix, where n is a positive integer, corresponding to the number of time points in the signal. In the example here, the matrix is a 1×401 matrix.

Set 80A is included in hidden layers 102, and is herein assumed to comprise at least one convolution layer. In the illustrated example a first hidden layer of the set comprises a 1D convolution layer 104, which consists of at least one filter, or kernel, configured to perform the convolution of the layer by scanning across the values derived from input layer 100. Each kernel is a 1D filter, and the illustrated example depicts layer 104 comprising a first 1D kernel 108 and a second 1D kernel 112, each kernel having five elements formed as a 1×5 matrix. Typically, each kernel of the layer may have fewer or more elements than five, and in one example kernels have three elements. Also, while the illustration assumes the convolution layer has two kernels, there are typically more than two kernels.

Kernels in the convolution layers, such as in layer 104, are typically configured, by their convolution operation, to filter or isolate a feature of the data being analyzed. Typical features isolated by a kernel are a region comprising a negative slope, or a region comprising high values. The kernels operate by sliding, in a step manner with a preset stride, along a presented set of data, and forming convolutions of the section of data “covered” by the kernel after each step. Typically padding elements are added to the initial and terminal elements of the convolution layers to ensure that the convolution of these elements by the kernels is correct.

Convolution layer 104 is typically followed by a 1D pooling layer 116 which is configured to receive the output of layer 104. Pooling layer 116 in turn comprises at least one 1D filter, associated with the layer, that is configured to reduce the data generated by each of the kernels of convolution layer 104. By way of example, layer 116 is assumed to have two 1D filters 120, 122 of two elements each, formed as 1×2 matrices that are applied to data received from convolution layer 104. The filters of the pooling layer do not perform convolution, but rather are configured to reduce the data from layer 104, for example by taking the maximum value of the filtered data, in which case pooling layer 116 is a max-pooling layer.

Hidden layers 102 of set 80A may comprise further convolution layers followed by pooling layers, such as illustrated convolution layer 124 followed by pooling layer 132. Convolution layer 124 is structured and operates substantially as convolution layer 104; pooling layer 132 is structured and operates substantially as pooling layer 116.

Data from the convolution layers of set 80A is transferred to one or more fully connected layers within hidden layers 102, herein assumed to comprise a fully connected output layer 140 of the set. Fully connected layer 140 is in turn connected to fully connected concatenation layer 96, so that results 80AR from layer 140, herein termed interim results, transfer to the concatenation layer. Output layer 140 typically comprises the same number of neurons as input layer 100, i.e., 401.

In an example of the present disclosure, layer set 84 and layer set 88 have respective examples set 84A and set 88A that are structurally substantially similar to, and operate in a substantially similar manner, to example 80A. As is described herein, the three layer sets are trained using different input signals, i.e., set 80 is trained using 30A^{V}R, set 84 is trained using 30B^{V}R, and set 88 is trained using 30AV30B Thus, while the input signals for the three sets may have the same structure of a 1x401 matrix, (and will also have the same LAT) in response to the training the three sets typically have different weights associated with their neurons and kernels. Also, in response to the training, there may be different numbers and different dimensions of the layers and the kernels in the three sets. Sets 84A and 88A have respective interim results 84AR and 88AR. It will be understood that the interim results, 80AR, 84AR, 88AR are typically different.

The similarity of the structure and operation of the three sets is illustrated in FIG. 3 by adding to identifiers 80A, 30A^{V}R, 80AR respective identifiers, in brackets, 84A, 30B^{V}R, 84AR, and 88A, 30A^{V}30B, 88AR.

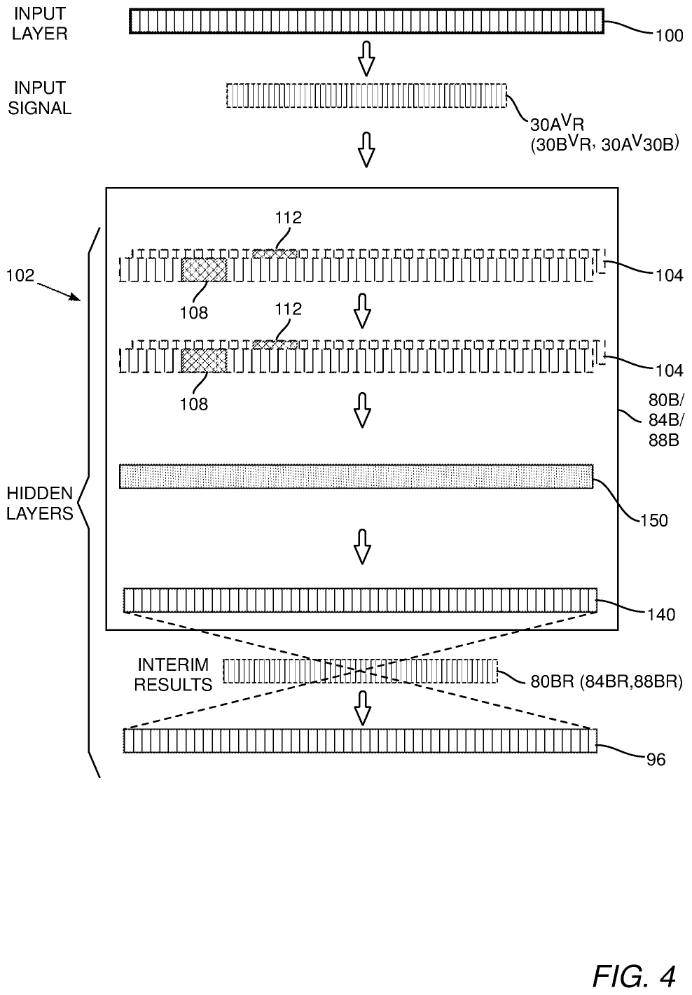

FIG. 4 is a schematic diagram of an alternative example of set of layers 80, herein termed set 80B. Apart from the differences described below, the operation of set 80B is generally similar to that of set 80A (FIG. 3), and elements indicated by the same reference numerals in both sets are generally similar in construction and in operation.

In contrast to set 80A, set 80B does not use pooling layers; rather, data from the convolutional layers, herein assumed to comprise two such layers 104, is transferred to a 1D flatten layer 150. As for set 80A, set 80B comprises fully connected layer 140, which receives data from flatten layer 150, and which is in turn connected to fully connected concatenation layer 96, so that interim results 80BR from layer 140 transfer to the concatenation layer.

In an alternative example of the present disclosure, set 84 and set 88 have respective examples set 84B and set 88B that, except for the differences referred to above regarding examples 80A, 84A, and 88A, are structurally substantially similar to, and operate in a substantially similar manner, to example 80B. Sets 84B and 88B have respective interim results 8BAR and 88BR.

The similarity of the structure and operation of the three sets is illustrated in FIG. 4 by adding to identifiers 80B, 30A^{V}R, 80BR respective identifiers, in brackets, 84B, 30B^{V}R, 84BR, and 88B, 30A^{V}30B, 88BR.

The above description for the three sets of layers 80, 84, and 88 illustrates that their structures are substantially the same. While this is generally the case for set of layers 92, there are differences between set 92 and the three sets 80, 84, and 88, as is explained below.

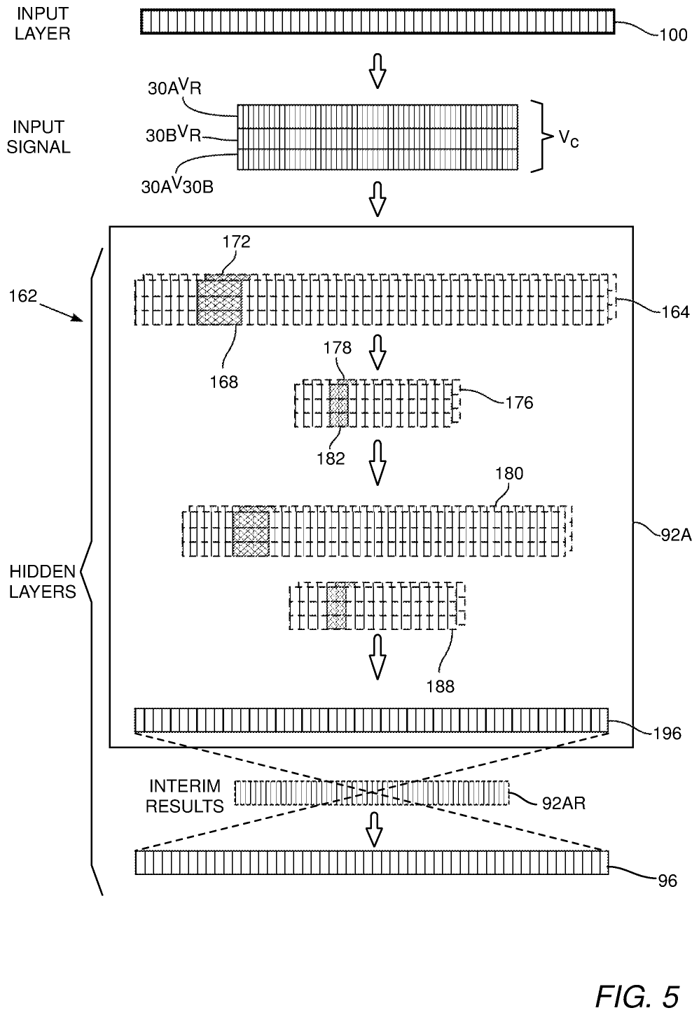

FIG. 5 is a schematic diagram of an example of set 92, herein termed set 92A. Apart from the differences described below, the operation of set 92A is generally similar to that of set 80A (FIG. 3), and elements indicated by the same reference numerals in both sets are generally similar in construction and in operation.

However, in contrast to sets 80, 84, and 88, which have as their input a signal in the form of a 1×n matrix, set 92A is configured to receive from input layer 100 a signal V_{C} in the form of a two-dimensional (2D) 3×n matrix, wherein, in the example described herein, n=401. Signal V_{C} is formed as a combination of 30A^{V}R, 30B^{V}R, and 30AV30B Set 92A comprises hidden layers 162, which are generally similar to hidden layers 102, comprising at least one convolutional layer. Signal V_{C} is input to a first hidden layer of set 92A, and in the illustrated example a first hidden layer of the set comprises a 2D convolution layer 164, which consists of at least one filter, or kernel, configured to perform the convolution of the layer by scanning across the values derived from input layer 100.

Each kernel is a 2D filter, and the illustrated example depicts layer 164 as comprising a first 2D kernel 168 and a second 2D kernel 172, each kernel having five elements formed as a 3×5 matrix. Typically, each kernel of the layer, while having a depth of three, may have as its width fewer or more elements than five, and in one example kernels have three elements. Also, while the illustration assumes the convolution layer has two kernels, there are typically more than two kernels.

Convolution layer 164 is, by way of example, followed by a 2D pooling layer 176 which is configured to receive the output of layer 164. Pooling layer 176 comprises at least one 2D filter, associated with the layer, that is configured to reduce the data generated by each of the kernels of convolution layer 164. As for the associated convolution layer kernels, each filter of the pooling layer has a depth of three. By way of example, layer 176 is assumed to have two 2D filters 178, 182 of two elements each, formed as 3×2 matrices that are applied to data received from convolution layer 164.

Hidden layers 162 may comprise further 2D convolution layers followed by 2D pooling layers, such as illustrated convolution layer 180, which has 2D kernels with a depth of three. As stated, the convolution layers are followed by pooling layers, such as illustrated pooling layer 188, which have 2D filters with a depth of three. Convolution layer 180 is structured and operates substantially as convolution layer 164; pooling layer 188 is structured and operate substantially as pooling layer 176.

Data from the convolution layers is transferred to one or more fully connected layers within hidden layers 162, herein assumed to comprise a fully connected output layer 196. Output layer 196 typically comprises the same number of neurons as input layer 100, i.e., 401, each neuron being configured to accept inputs having a depth of three. Interim results 92AR from output layer 196 are transferred to concatenation layer 96.

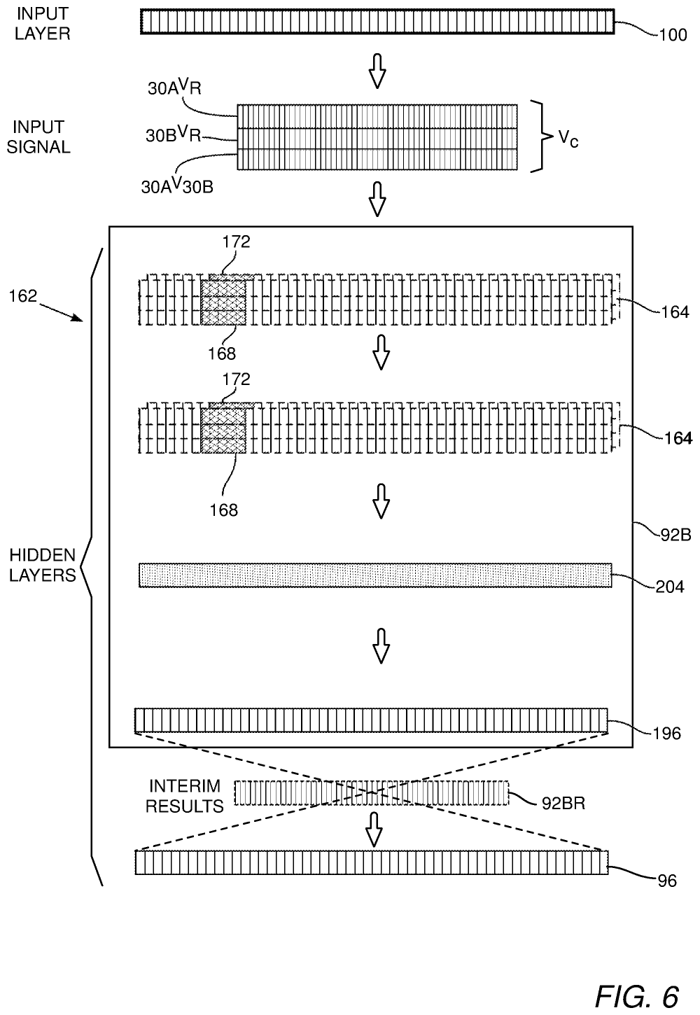

FIG. 6 is a schematic diagram of an alternative example of set of layers 92, herein termed set 92B. Apart from the differences described below, the operation of set 92B is generally similar to that of set 92A (FIG. 5), and elements indicated by the same reference numerals in both sets are generally similar in construction and in operation.

Set 92B does not use pooling layers; rather, data from the convolutional layers, herein assumed to comprise two such layers 164, is transferred to a 1D flatten layer 204. As for set 92A, set 92B comprises fully connected output layer 196, which receives data from flatten layer 204, and which provides interim results 92BR to concatenation layer 96.

Returning to FIG. 2, the interim results from sets of layers 80, 84, 88, and 92 are all input to concatenation layer 96, as is described above. Concatenation layer 96 feeds to fully connected output layer 144, which has 401 neurons, each of which corresponds to a time point, and the value provided by the neuron typically provides a probability that the time point corresponds to the LAT of the input signal. The output derived from the output layer may be expressed as one-hot values, and typically there is a single one-hot value of one, corresponding to the time point that ANN 34 assigns to the LAT of the input signal.

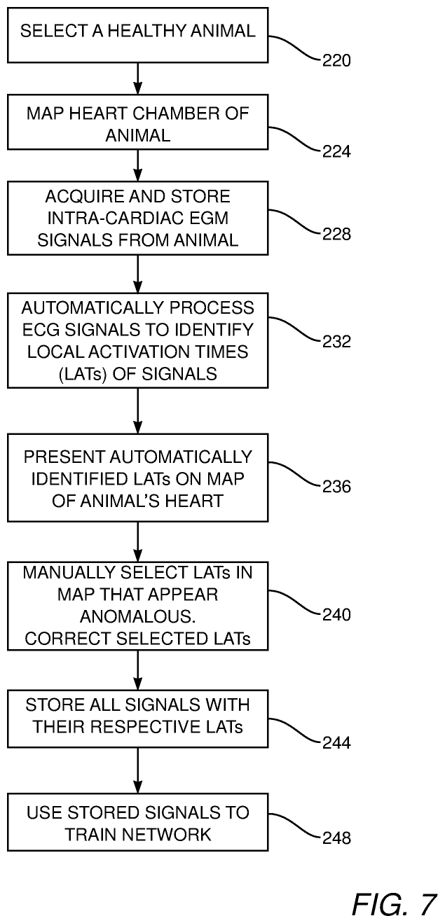

As stated above, prior to being used in the procedure illustrated in FIG. 1, sets 80, 84, 88, and 92, are trained, using stored intracardiac EGM (IEGM) signals, together with respective values of the LAT associated with the signals. FIG. 7 is a flowchart illustrating a process for producing such signals and LAT values, and the steps of the flowchart are described below.

FIG. 7 is a diagram of a flowchart of an algorithm for generating a corpus of samples that may be used in training the artificial neutral network of the present disclosure. A large number of sample IEGM signals are required in order that the training is effective, and the correct LAT is needed for each signal. As stated above, there is a problem in using most of the signals that are available, since these are typically from unhealthy patients, and such data may have incorrect LAT values. The algorithm described hereinbelow solves this problem.

By way of example, the algorithm is assumed to be implemented using elements illustrated in FIG. 1, e.g., processor 32, memory 31, probe 26, tracking module 60, and position sensors 61. However, it will be understood that the algorithm may be implemented using any other convenient processor, memory, probe, and elements for tracking the probe.

In an initial step 220, a healthy animal, typically a pig, is chosen, and catheter probe 26 is inserted into the heart of the animal, so as to contact a wall of one of the heart chambers. The catheter is assumed to have two electrodes 30 that contact the wall, and an indifferent electrode, such as patch 65, is attached to the skin of the animal.

In a mapping step 224, the catheter is moved within the heart chamber, and tracking module 60 processes signals from sensors 61 in order to generate a map of the chamber.

In an acquisition step 228, which may be implemented simultaneously with step 224, unipolar IEGM signals from the two catheter electrodes measured with respect to the indifferent electrode, as well as a bipolar signal between the electrodes, are acquired by the processor. At the same time, the processor uses tracking module 60 and sensors 61 to determine the position of the electrodes. The signals and their electrode positions are stored in the memory. It will be understood that the signals correspond to the three types of signals referred to above, i.e., 30A^{V}R, 30B^{V}R, and 30A^{V}30B.

In an automatic processing step 232, the processor implements an algorithm for automatically analyzing each of the signals, and from the analysis assigning a respective value of the LAT associated with each signal. One algorithm that may be implemented is the wavefront annotation algorithm, described in U.S. Pat. No. 11,058,342, but the processor may use any other suitable algorithm.

In a presentation step 236, the processor presents on display screen 44 a map of the heart chamber, such as map 68, overlaid by the LAT values that have been calculated in step 232. The overlaid LAT values are typically displayed as a spectrum of different colors; for example an LAT of 100 ms may display as red, and an LAT of 300 ms may display as blue.

In a correction step 240, an algorithm operator, who may typically have expertise substantially less than that of physician 22, inspects the displayed map. Because the IEGM signals are acquired from a healthy animal, the LATs of the animal heart typically change in a smooth, gradual manner. Consequently, the LAT colors presented in the displayed map should also change in a smooth, gradual, manner, and any deviations from this gradual color change are easily visible to the algorithm operator.

For example, within a red region of the map there may be an orange point; such a point corresponds to a signal where the algorithm calculating the LAT has found an LAT that is different from LAT values of its surroundings. However, because the signals are from a healthy animal, the different color is an anomaly which must be caused by an algorithm error.

The algorithm operator may select an anomalous point. Once selected, a correction to the LAT of the point may be applied by any convenient method, which may be manual or automatic. For example, if screen 44 is configured as a touch screen, the operator may touch the anomalous point, and processor 32 may be configured to automatically reassign the LAT of the point to a value corresponding to the surrounding LATs. Alternatively, the operator may input a reassigned LAT value for the point. The processor may then save the signal generating the LAT, and the reassigned LAT value.

In a storage step 244, once the algorithm operator has completed step 220, the operator may store all the signals and their respective LAT values used for the map. It will be understood that the stored signals and LAT values comprise a corpus that includes those signals where the operator re-assigned the LAT values, as well as the signals (and their automatically found LAT value) wherein the operator did not re-assign the LAT. It will be understood that considering the set of all the signals analysed, the subset of signals that have reassigned LATs, and the subset of signals that do not have reassigned LATs, are complements of each other.

The stored signals may then be used, in a training step 248, to train ANN 34.

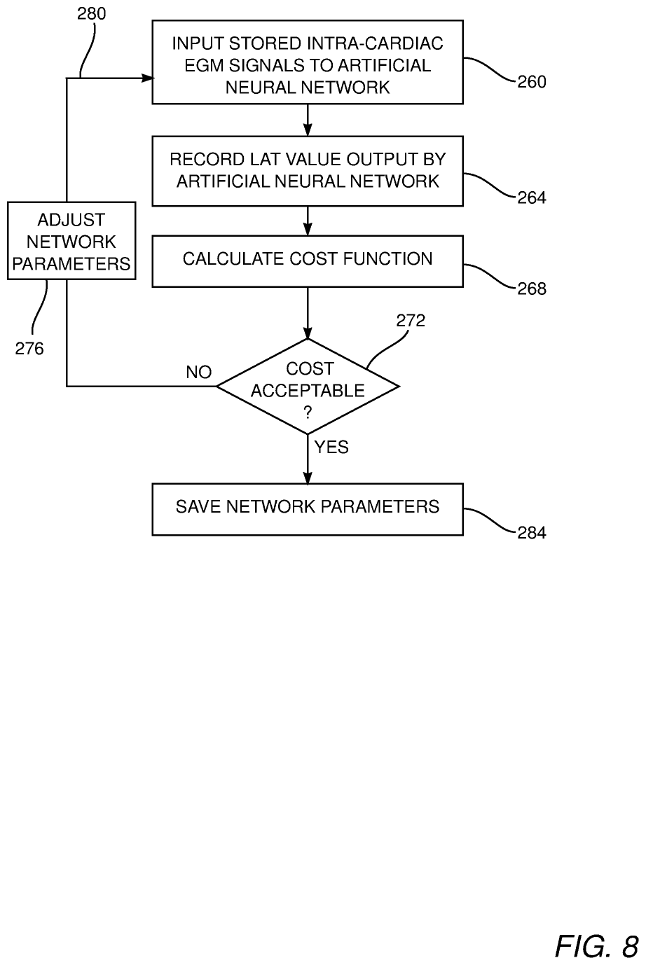

FIG. 8 is a flowchart of an algorithm for training ANN 34. The training is an iterative process, wherein parameters of the network, such as the weights of the network neurons, the sizes and weights of the filters of the convolutional layers, and the number and type of the different layers, are adjusted so as to optimize the output of the network. The training is assumed to be performed using processor 32, but any other suitable processor may be used.

In an initial step 260 a corpus of IEGM signals, that may have been produced using the algorithm of FIG. 7, is input to input layer 100. Because of the large number of signals that are in the corpus, the input of the signals is typically implemented as a batch process.

In an evaluation step 264, the processor records the LAT values calculated and output by the network, and in a cost calculation step 268 the processor, using the known LAT values of the input batch of signals, calculates a cost relating the known LAT values to the LAT values output by the network.

Step 268 may use any cost function known in the art, such as a quadratic cost function or a cross-entropy cost function.

In a decision step 272 the processor determines if the cost calculated in step 268 is low enough, so as to be acceptable. If the cost is too high, i.e., the cost is not acceptable so that the decision returns negative, the processor, in an adjustment step 276, alters the parameters of the network using any suitable optimization algorithm, for example, a gradient descent algorithm such as the Adam optimizer.

As stated above, and as illustrated by arrow 280, the training process is an iterative process, so that from step 276 the processor returns to initial step 260.

If decision step 272 returns positive, i.e., the cost calculated in step 268 is sufficiently low, then in a save step 284 the processor saves the network parameters that have been calculated, and uses these in ANN 34.

As used herein, the terms “about” or “approximately” for any numerical values or ranges of an entity indicate a suitable dimensional tolerance that allows the entity to function for its intended purpose as described herein. More specifically, “about” or “approximately” may refer to the range of values ±10% of the recited value, e.g. “about 80%” may refer to the range of values from 72% to 88.

### EXAMPLES

Example 1. A method for medical diagnosis, comprising:

receiving electrophysiological data comprising first intracardiac electrogram (IEGM) signals acquired by a first pair of electrodes comprising a first intracardiac electrode and second IEGM signals acquired by a second pair of electrodes comprising a second intracardiac electrode in proximity to the first intracardiac electrode;

inputting the first and second IEGM signals respectively to a first convolutional layer of a neural network and a second convolutional layer parallel to the first convolutional layer in the neural network to generate respective first and second interim results;

inputting the first and second interim results together into one or more common layers of the neural network; and

receiving from an output layer of the neural network, responsively to inputting the first and second interim results, an indication of a local activation time at a location of the first and second intracardiac electrodes.

Example 2. The method according to example 1, wherein the first and second pairs of electrodes have a common indifferent electrode.

Example 3. The method according to example 2, wherein at least one of the first and second signals comprises unipolar signals measured relative to the indifferent electrode.

Example 4. The method according to example 2, wherein the first signals comprise a combination of first unipolar signals generated between the first intracardiac electrode and the indifferent electrode, second unipolar signals generated between the second intracardiac electrode and the indifferent electrode, and bipolar signals generated between the first intracardiac electrode and the second intracardiac electrode.

Example 5. The method according to example 1, wherein the first signals are in the form of one-dimensional (1D) matrices and the first convolutional layer is one dimensional with at least one 1D kernel configured to slide along the first convolutional layer.

Example 6. The method according to example 5, where the neural network comprises at least one 1D convolutional layer subsequent to the first convolutional layer and prior to the one or more common layers.

Example 7. The method according to example 5, where the neural network comprises at least one 1D fully connected layer subsequent to the first convolutional layer and prior to the one or more common layers.

Example 8. The method according to example 1, where the neural network comprises at least one flatten layer subsequent to the first convolutional layer and prior to the one or more common layers.

Example 9. The method according to example 1, wherein the first signals are in the form of two-dimensional (2D) matrices and the first convolutional layer is two dimensional with at least one 2D kernel configured to slide along the first convolutional layer.

Example 10. The method according to example 9, where the neural network comprises at least one 2D convolutional layer subsequent to the first convolutional layer and prior to the one or more common layers.

Example 11. The method according to example 9, where the neural network comprises at least one 2D fully connected layer subsequent to the first convolutional layer and prior to the one or more common layers.

Example 12. The method according to example 1, wherein the one or more common layers comprise at least one fully connected layer.

Example 13. A method, comprising:

estimating respective local activation times (LATs) of a first set of intracardiac electrocardiogram signals;

generating a three-dimensional (3D) map based on the estimated LATs;

receiving an indication of anomalies in the 3D map;

using the anomalies and regions of the 3D map not including the anomalies to generate training data;

using the training data to train an artificial neural network; and

applying the artificial neural network to estimate LATs of a second set of intracardiac electrocardiogram signals.

Example 14. The method according to example 13, wherein the first set of intracardiac electrocardiogram signals are generated from a healthy animal.

Example 15. The method according to example 13, wherein estimating the respective LATs of the first set is implemented automatically by a computer processor.

Example 16. The method according to example 13, wherein the indication is provided by manual inspection of the 3D map.

Example 17. The method according to example 16, and comprising, in response to the manual inspection, a computer processor automatically re-assigning respective LATs to the signals corresponding to the anomalies.

Example 18. The method according to example 16, and comprising, in response to the manual inspection, manually re-assigning respective LATs to the signals corresponding to the anomalies.

Example 19. The method according to example 13, wherein the training data comprises the first set of signals, LATs corresponding to the anomalies, and LATs corresponding to the regions of the 3D map not including the anomalies.

Example 20. Apparatus for medical diagnosis, comprising:

a probe (26) comprising a first intracardiac electrode (30A) in proximity to a second intracardiac electrode (30B);

a neural network (34) comprising:

a first convolutional layer (80) and a second convolutional layer (84) parallel to the first convolutional layer;

one or more common layers; and

an output layer; and

a processor (32), configured to:

receive electrophysiological data comprising first intracardiac electrogram (IEGM) signals acquired by a first pair of electrodes comprising the first intracardiac electrode and second IEGM signals acquired by a second pair of electrodes comprising the second intracardiac electrode;

input the first and second IEGM signals respectively to the first convolutional layer and the second convolutional layer to generate respective first and second interim results;

input the first and second interim results together into the one or more common layers; and

receive from the output layer, responsively to inputting the first and second interim results, an indication of a local activation time at a location of the first and second intracardiac electrodes.

Example 21. The apparatus according to example 20, and comprising an indifferent electrode common to the first and second pairs of electrodes.

Example 22. The apparatus according to example 21, wherein at least one of the first and second signals comprises unipolar signals measured relative to the indifferent electrode.

Example 23. The apparatus according to example 21, wherein the first signals comprise a combination of first unipolar signals generated between the first intracardiac electrode and the indifferent electrode, second unipolar signals generated between the second intracardiac electrode and the indifferent electrode, and bipolar signals generated between the first intracardiac electrode and the second intracardiac electrode.

Example 24. The apparatus according to example 21, wherein the first signals are in the form of one-dimensional (1D) matrices and the first convolutional layer is one dimensional with at least one 1D kernel configured to slide along the first convolutional layer.

Example 25. The apparatus according to example 24, 15 where the neural network comprises at least one 1D convolutional layer subsequent to the first convolutional layer and prior to the one or more common layers.

Example 26. The apparatus according to example 24, where the neural network comprises at least one 1D fully connected layer subsequent to the first convolutional layer and prior to the one or more common layers.

Example 27. The apparatus according to example 21, where the neural network comprises at least one flatten layer subsequent to the first convolutional layer and prior to the one or more common layers.

Example 28. The apparatus according to example 21, wherein the first signals are in the form of two-dimensional (2D) matrices and the first convolutional layer is two dimensional with at least one 2D kernel configured to slide along the first convolutional layer.

Example 29. The apparatus according to example 28, where the neural network comprises at least one 2D convolutional layer subsequent to the first convolutional layer and prior to the one or more common layers.

Example 30. The apparatus according to example 28, where the neural network comprises at least one 2D fully connected layer subsequent to the first convolutional layer and prior to the one or more common layers.

Example 31. The apparatus according to example 21, wherein the one or more common layers comprise at least one fully connected layer.

Example 32. Apparatus, comprising:

a display (44);

an artificial neural network (34); and

a processor (32) configured to:

estimate respective local activation times (LATs) of a first set of intracardiac electrocardiogram signals;

generate a three-dimensional (3D) map based on the estimated LATs and present the 3D map on the display;

receive an indication of anomalies in the 3D map;

use the anomalies and regions of the 3D map not including the anomalies to generate training data;

use the training data to train the artificial neural network; and

apply the artificial neural network to estimate LATs of a second set of intracardiac electrocardiogram signals.

Example 33. The apparatus according to example 32, wherein the first set of intracardiac electrocardiogram signals are generated from a healthy animal.

Example 34. The apparatus according to example 32, wherein estimating the respective LATs of the first set is implemented automatically by the processor.

Example 35. The apparatus according to example 32, wherein the indication is provided by manual inspection of the 3D map.

Example 36. The apparatus according to example 35, and comprising, in response to the manual inspection, the processor automatically re-assigning respective LATs to the signals corresponding to the anomalies.

Example 37. The apparatus according to example 35, and comprising, in response to the manual inspection, manually re-assigning respective LATs to the signals corresponding to the anomalies.

Example 38. The apparatus according to example 32, wherein the training data comprises the first set of signals, LATs corresponding to the anomalies, and LATs corresponding to the regions of the 3D map not including the anomalies.

It will be appreciated that the examples described above are cited by way of example, and that the present disclosure is not limited to what has been particularly shown and described hereinabove. Rather, the scope of the present disclosure includes both combinations and subcombinations of the various features described hereinabove, as well as variations and modifications thereof which would occur to persons skilled in the art upon reading the foregoing description and which are not disclosed in the prior art.

1. A method for medical diagnosis, comprising:
receiving electrophysiological data comprising first intracardiac electrogram (IEGM) signals acquired by a first pair of electrodes comprising a first intracardiac electrode and second IEGM signals acquired by a second pair of electrodes comprising a second intracardiac electrode in proximity to the first intracardiac electrode;
inputting the first and second IEGM signals respectively to a first convolutional layer of a neural network and a second convolutional layer parallel to the first convolutional layer in the neural network to generate respective first and second interim results;
inputting the first and second interim results together into one or more common layers of the neural network; and
receiving from an output layer of the neural network, responsively to inputting the first and second interim results, an indication of a local activation time at a location of the first and second intracardiac electrodes.

2. The method according to claim 1, wherein the first and second pairs of electrodes have a common indifferent electrode.

3. The method according to claim 2, wherein at least one of the first and second signals comprises unipolar signals measured relative to the indifferent electrode.

4. The method according to claim 2, wherein the first signals comprise a combination of first unipolar signals generated between the first intracardiac electrode and the indifferent electrode, second unipolar signals generated between the second intracardiac electrode and the indifferent electrode, and bipolar signals generated between the first intracardiac electrode and the second intracardiac electrode.

5. The method according to claim 1, wherein the first signals are in the form of one-dimensional (1D) matrices and the first convolutional layer is one dimensional with at least one 1D kernel configured to slide along the first convolutional layer.

6. The method according to claim 5, where the neural network comprises at least one 1D convolutional layer subsequent to the first convolutional layer and prior to the one or more common layers.

7. The method according to claim 5, where the neural network comprises at least one 1D fully connected layer subsequent to the first convolutional layer and prior to the one or more common layers.

8. The method according to claim 1, where the neural network comprises at least one flatten layer subsequent to the first convolutional layer and prior to the one or more common layers.

9. The method according to claim 1, wherein the first signals are in the form of two-dimensional (2D) matrices and the first convolutional layer is two dimensional with at least one 2D kernel configured to slide along the first convolutional layer.

10. The method according to claim 9, where the neural network comprises at least one 2D convolutional layer subsequent to the first convolutional layer and prior to the one or more common layers.

11. The method according to claim 9, where the neural network comprises at least one 2D fully connected layer subsequent to the first convolutional layer and prior to the one or more common layers.

12. The method according to claim 1, wherein the one or more common layers comprise at least one fully connected layer.

13. A method, comprising:
estimating respective local activation times (LATs) of a first set of intracardiac electrocardiogram signals;
generating a three-dimensional (3D) map based on the estimated LATs;
receiving an indication of anomalies in the 3D map;
using the anomalies and regions of the 3D map not including the anomalies to generate training data;
using the training data to train an artificial neural network; and
applying the artificial neural network to estimate LATs of a second set of intracardiac electrocardiogram signals.

14. The method according to claim 13, wherein the first set of intracardiac electrocardiogram signals are generated from a healthy animal.

15. The method according to claim 13, wherein estimating the respective LATs of the first set is implemented automatically by a computer processor.

16. The method according to claim 13, wherein the indication is provided by manual inspection of the 3D map.

17. The method according to claim 16, and comprising, in response to the manual inspection, a computer processor automatically re-assigning respective LATs to the signals corresponding to the anomalies.

18. The method according to claim 16, and comprising, in response to the manual inspection, manually re-assigning respective LATs to the signals corresponding to the anomalies.

19. The method according to claim 13, wherein the training data comprises the first set of signals, LATs corresponding to the anomalies, and LATs corresponding to the regions of the 3D map not including the anomalies.

20. Apparatus for medical diagnosis, comprising:
a probe comprising a first intracardiac electrode in proximity to a second intracardiac electrode;
a neural network comprising:
a first convolutional layer and a second convolutional layer parallel to the first convolutional layer;
one or more common layers; and
an output layer; and
a processor, configured to:
receive electrophysiological data comprising first intracardiac electrogram (IEGM) signals acquired by a first pair of electrodes comprising the first intracardiac electrode and second IEGM signals acquired by a second pair of electrodes comprising the second intracardiac electrode;
input the first and second IEGM signals respectively to the first convolutional layer and the second convolutional layer to generate respective first and second interim results;
input the first and second interim results together into the one or more common layers; and
receive from the output layer, responsively to inputting the first and second interim results, an indication of a local activation time at a location of the first and second intracardiac electrodes.

21. The apparatus according to claim 20, and comprising an indifferent electrode common to the first and second pairs of electrodes.

22. The apparatus according to claim 21, wherein at least one of the first and second signals comprises unipolar signals measured relative to the indifferent electrode.

23. The apparatus according to claim 21, wherein the first signals comprise a combination of first unipolar signals generated between the first intracardiac electrode and the indifferent electrode, second unipolar signals generated between the second intracardiac electrode and the indifferent electrode, and bipolar signals generated between the first intracardiac electrode and the second intracardiac electrode.

24. The apparatus according to claim 21, wherein the first signals are in the form of one-dimensional (1D) matrices and the first convolutional layer is one dimensional with at least one 1D kernel configured to slide along the first convolutional layer.

25. The apparatus according to claim 24, where the neural network comprises at least one 1D convolutional layer subsequent to the first convolutional layer and prior to the one or more common layers.

26. The apparatus according to claim 24, where the neural network comprises at least one 1D fully connected layer subsequent to the first convolutional layer and prior to the one or more common layers.

27. The apparatus according to claim 21, where the neural network comprises at least one flatten layer subsequent to the first convolutional layer and prior to the one or more common layers.

28. The apparatus according to claim 21, wherein the first signals are in the form of two-dimensional (2D) matrices and the first convolutional layer is two dimensional with at least one 2D kernel configured to slide along the first convolutional layer.

29. The apparatus according to claim 28, where the neural network comprises at least one 2D convolutional layer subsequent to the first convolutional layer and prior to the one or more common layers.

30. The apparatus according to claim 28, where the neural network comprises at least one 2D fully connected layer subsequent to the first convolutional layer and prior to the one or more common layers.

31. The apparatus according to claim 21, wherein the one or more common layers comprise at least one fully connected layer.

32. Apparatus, comprising:
a display;
an artificial neural network; and
a processor configured to:
estimate respective local activation times (LATs) of a first set of intracardiac electrocardiogram signals;
generate a three-dimensional (3D) map based on the estimated LATs and present the 3D map on the display;
receive an indication of anomalies in the 3D map;
use the anomalies and regions of the 3D map not including the anomalies to generate training data;
use the training data to train the artificial neural network; and
apply the artificial neural network to estimate LATs of a second set of intracardiac electrocardiogram signals.

33. The apparatus according to claim 32, wherein the first set of intracardiac electrocardiogram signals are generated from a healthy animal.

34. The apparatus according to claim 32, wherein estimating the respective LATs of the first set is implemented automatically by the processor.

35. The apparatus according to claim 32, wherein the indication is provided by manual inspection of the 3D map.

36. The apparatus according to claim 35, and comprising, in response to the manual inspection, the processor automatically re-assigning respective LATs to the signals corresponding to the anomalies.

37. The apparatus according to claim 35, and comprising, in response to the manual inspection, manually re-assigning respective LATs to the signals corresponding to the anomalies.

38. The apparatus according to claim 32, wherein the training data comprises the first set of signals, LATs corresponding to the anomalies, and LATs corresponding to the regions of the 3D map not including the anomalies.
